(19) United States
(12) Patent Application Publication
(10) Pub. No.: US 2012/0101277 A1
(43) Pub. Date: Apr. 26, 2012

(54) CRYSTALLINE FORM OF POSACONAZOLE

(75) Inventors: Josef Wieser, Kundl (AT); Arthur Pichler, Kundl (AT); Andreas Hotter, Kundl (AT); Ulrich Griesser, Innsbruck (AT); Christoph Langes, Innsbruck (AT)

(73) Assignee: SANDOZ AG, Basel (CH)

(21) Appl. No.: 13/379,060

(22) PCT Filed: Jul. 9, 2010
(86) PCT No.: PCT/EP2010/059851

§ 371 Date: Dec. 19, 2011

(30) Foreign Application Priority Data
Jul. 9, 2009 (EP) 09165050.7

(51) Int. Cl. C07D 405/14; A61P 31/10

(52) U.S. Cl. 544/366

(57) ABSTRACT

The present invention relates to crystalline form II-S, its preparation and its use to prepare other crystalline forms of posaconazole, in particular crystalline form IV of posaconazole.

### FIELD OF THE INVENTION

The present invention relates to crystalline form II-S of posaconazole, to its preparation and to its use for preparing other crystalline forms of posaconazole such as crystalline form IV. Additionally, the present invention relates to an improved process for preparing said other crystalline forms of posaconazole, in particular of crystalline form IV. Crystalline forms of posaconazole can be used in pharmaceutical compositions to treat or prevent fungal infections.

### BACKGROUND OF THE INVENTION

Posaconazole (CAS Registry Number 171228-49-2; CAS Name: 2,5-anhydro-1,3,4-trideoxy-2-C-(2,4-difluorophenyl)-4-[[4-[4-[4-[1-[(1S,2S)-1-ethyl-2-hydroxypropyl]-1,5-dihydro-5-oxo-4H-1,2,4-triazol-4-yl]phenyl]-1-piperazinyl]phenoxy]methyl]-1-(1H-1,2,4-triazol-1-yl)-D-threo-pentitol) which is represented by the following general formula (I)

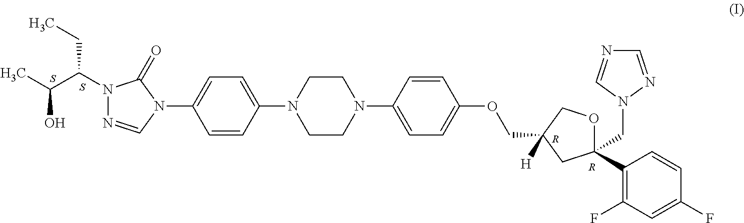

is known as an antifungal agent. It is available as an oral suspension (40 mg/ml) under the trademark NOXAFIL® from Schering Corporation, Kenilworth, N.J.

WO95/17407 and WO 96/38443 disclose the compound having the general formula (I) and its use in treating fungal infections.

Various pharmaceutical compositions comprising posaconazole and being adapted for oral, topical or parenteral use are described e.g. in WO 02/80678, U.S. Pat. No. 5,972,381, U.S. Pat. No. 5,834,472, U.S. Pat. No. 4,957,730 and WO 2005/117831.

As was mentioned above, WO 95/17407 and WO 96/38443 disclose the compound having the general formula (I). However, during prosecution of the subsequently filed European patent application no. 98951994.7, now European patent EP 1 021 439 B1, the applicant declared that the methods disclosed in these publications only lead to the compound of formula (I) as an amorphous solid.

Polymorphism is a phenomenon relating to the occurrence of different crystal forms for one molecule. There may be several different crystalline forms for the same molecule with distinct crystal structures and distinct and varying physical properties like melting point, XRPD pattern, IR-spectrum and solubility profile. These polymorphs are thus distinct solid forms which share the molecular formula of the compound from which the crystals are made up, however, they may have distinct advantageous physical properties which can have a direct effect on the ability to process and/or manufacture the drug product, like flowability, as well as physical properties such as solubility, stability and dissolution properties which can have a direct effect on drug product stability, solubility, dissolution, and bioavailability.

Three polymorphic forms of posaconazole designated as forms I, II and III are described and characterized in WO 99/18097 (U.S. Pat. No. 6,713,481, U.S. Pat. No. 6,958,337). Crystalline forms II and III were found to be unstable under the conditions investigated, so that crystalline form I was considered to be useful in the development of a pharmaceutical product.

The present inventors have disclosed further crystalline forms of posaconazole such as crystalline form IV in co-pending European patent application no. 08159600.9 which has improved properties when compared to form I. Form IV may be prepared from amorphous posaconazole or from crystalline form III, or alternatively from crystalline form I or II in which case the presence of seed crystals of form IV is required. In those processes, temperatures of at most 60° C. are applied, and the transformation of the starting materials to crystalline form IV takes from about 2 days to about 15 days at ambient temperatures in the absence of seed crystals. These processes may lead to formation of traces of un-wanted polymorphic by-products.

There remains a need for alternative polymorphic forms of posaconazole. Furthermore, there is a need of alternative polymorphic forms of posaconazole which are particularly suitable for the preparation of other polymorphic forms of posaconazole. Another need is the provision of an alternative way of preparing polymorphic form IV of posaconazole which is faster and leads to less formation of un-wanted by-products. Finally, it would be desired to provide an alternative way of preparing polymorphic form IV of posaconazole on a commercial scale.

### SUMMARY OF THE INVENTION

In one embodiment the present invention relates to crystalline form II-S of posaconazole.

Crystalline form II-S of posaconazole can be described by an X-ray powder diffraction pattern comprising peaks at 2-theta angles of about 2.6°, 7.1°, 9.5°, 15.0°, 17.4° and 21.5°. The typical precision of the 2-theta values is in the range of ±0.2°.

Alternatively, crystalline form II-S of posaconazole can be characterized by an attenuated total reflectance infrared spectrum comprising absorption bands at wavenumbers of about 3650 cm⁻¹, 3392 cm⁻¹, 2968 cm⁻¹, 1688 cm⁻¹, 1510 cm⁻¹, 1227 cm⁻¹, 1036 cm⁻¹, 946 cm⁻¹, 820 cm⁻¹ and 680 cm⁻¹. The typical precision of the wavenumber values is in the range of ±2 cm⁻¹.

A further method of describing crystalline form II-S is by differential scanning calorimetry (DSC). A typical differential scanning calorimetry (DSC) curve of crystalline form II-S can be obtained at a heating rate of 10° C./min (open pan). Typical thermograms of form II-S of posaconazole are shown in FIG. 3. The DSC curve of form II-S shows a significant dehydration endotherm between 25° C. and 112° C. with peaks at 96° C. and 111° C. followed by a exothermic peak at 120.2° C.; form II-S is melting at 171.4° C. with a Tₒₙₛₑₜ of 169° C. (10° C./minute, open pan).

Crystalline form II-S of posaconazole contains 0 to 2.0 moles water per mole posaconazole and is thus a non-stoichiometric hydrate.

In another embodiment, the present invention relates to a process for preparing crystalline form II-S of posaconazole which process comprises the steps of
(a) admixing posaconazole and acetone in a concentration of posaconazole of about 1 g per 10 ml of acetone and heating the obtained mixture under reflux,
(b) adding water to the mixture obtained in step (a) while maintaining at reflux temperature to obtain a solution which contains acetone and water in a volume/volume (v/v) ratio of 10:3,
(c) cooling the solution obtained in step (b) to 0° C.-5° C. within a time period of about 1 to 2 hours to obtain a suspension, and
(d) isolating the solid product formed in the suspension obtained in step (c) by filtration.

Optionally, the hot solution obtained in step (b) whilst having a temperature of about 30 to 40° C., may be seeded with small amounts of the crystalline form II-S of posaconazole.

In a further embodiment, the present invention relates to the use of crystalline form II-S for the preparation of another crystalline form of posaconazole, preferably for the preparation of form IV of posaconazole. Another crystalline form of posaconazole is understood to mean crystalline forms I, II, III, Y and in particular form IV of posaconazole.

Thus, the present invention also relates to a process for preparing crystalline form IV of posaconazole comprising the steps of:
(a′) providing a suspension or dispersion of:
   (i) posaconazole, wherein posaconazole is selected from crystalline form II-S of posaconazole; and
   (ii) a mixture of water and methanol; and
(b′) allowing the posaconazole to transform to crystalline form IV at a temperature of at most 50° C.

If desired, seed crystals of crystalline form IV of posaconazole can be employed in this process.

Crystalline form II-S may be thus used to prepare another crystalline form of posaconazole, such as crystalline form II of posaconazole, and in particular crystalline form IV of posaconazole, which may further be used as a medicament to treat or prevent a fungal infection.

In the present invention the terms “suspension” and “dispersion” are intended to cover all types of mixtures of solid particles and liquids.

Other objects, features, advantages and aspects of the present invention will become apparent to those of skill from the following description. It should be understood, however, that the description and the following specific examples, while indicating preferred embodiments of the invention, are given by way of illustration only. Various changes and modifications within the spirit and scope of the disclosed invention will become readily apparent to those skilled in the art from reading the description and the other parts of the present disclosure.

### BRIEF DESCRIPTION OF THE DRAWINGS

FIG. 1: X-ray powder diffraction pattern of form II-S of posaconazole. In this figure the counts per 80 seconds are presented on the y-axis, while the 2 theta values in degrees are presented on the x-axis.

FIG. 2: Infrared spectrum of form II-S of posaconazole. The transmittance in % is plotted versus the wavenumber in cm⁻¹.

FIG. 3: Thermogravimetric and differential scanning calorimetric curve of form II-S of posaconazole. The temperature in ° C. is shown on the x-axis. The heat flow in mW is shown on the left hand ordinate (lower curve), while the mass loss in % is shown on the right hand ordinate (upper curve).

FIG. 4: Moisture sorption isotherm of form II-S of posaconazole. The water content in % (left hand ordinate) and the mol ratio of water (right hand ordinate) are plotted versus the relative humidity in %. The dotted line refers to desorption, while the solid line refers to sorption.

### DETAILED DESCRIPTION OF THE INVENTION

The present invention relates to crystalline form II-S of posaconazole.

Posaconazole is represented by the following general formula (I)

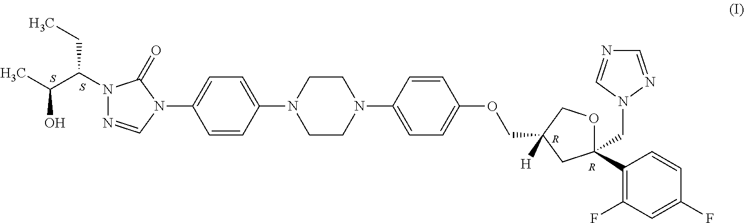

In the course of crystallization experiments carried out on posaconazole, the present inventors have found that a new crystalline form of posaconazole, designated as crystalline form II-S of posaconazole, can be prepared by crystallizing posaconazole using a solvent system such as acetone-water in a volume/volume (v/v) ratio of 10:3.

This is finding is surprising, because U.S. Pat. No. 6,958,337 discloses that crystalline form II is formed when crystallizing posaconazole using such a solvent system. In contrast to the process as described in U.S. Pat. No. 6,958,337, the present inventors have found that—when using the above described solvent system consisting of acetone and water in a volume/volume (v/v) ratio of 10:3—under the specific conditions described for the present invention, posaconazole does not crystallize as form II, but crystallizes in another pure polymorphous crystalline form, namely crystalline form II-S of posaconazole, which is different from form II and from any other known crystalline form.

Thus, crystalline form II-S of posaconazole can be prepared by crystallization of posaconazole using a mixture of acetone and water in a process that comprises the following steps:
(a) admixing posaconazole and acetone in a concentration of posaconazole of about 1 g per 10 ml of acetone and heating the obtained mixture under reflux,
(b) adding water to the mixture obtained in step (a) while maintaining at reflux temperature to obtain a solution which contains acetone and water in a volume/volume (v/v) ratio of 10:3,
(c) cooling the solution obtained in step (b) to 0° C.-5° C. within a time period of about 1 to 2 hours to obtain a suspension, and
(d) isolating the solid product formed in the suspension obtained in step (c) by filtration.

Optionally, the hot solution obtained in step (b)—when having a temperature of about 30 to 40° C.—may be seeded with small amounts of the crystalline form II-S of posaconazole.

The cool down of the hot solution obtained in step (b) to a temperature of about 0° C.-5° C. may made over a time period of about 1-2 hours, e.g. of 1.2-1.8 hours, e.g. of about 1.5 hours.

In a preferred embodiment the cooling rate of the solution from the starting temperature to about 30° C. is faster then the cooling rate from said about 30° C. to the final temperature.

As starting material described in step (a) any form of posaconazole can be used, such as amorphous posaconazole or crystalline posaconazole, e.g. crystalline forms I, II or III of posaconazole, or mixtures thereof.

Amorphous posaconazole can be obtained as described in WO 95/17407 and WO 96/38443. Crystalline forms I, II and III can be prepared as described in WO 99/18097, U.S. Pat. No. 6,713,481 or U.S. Pat. No. 6,958,337.

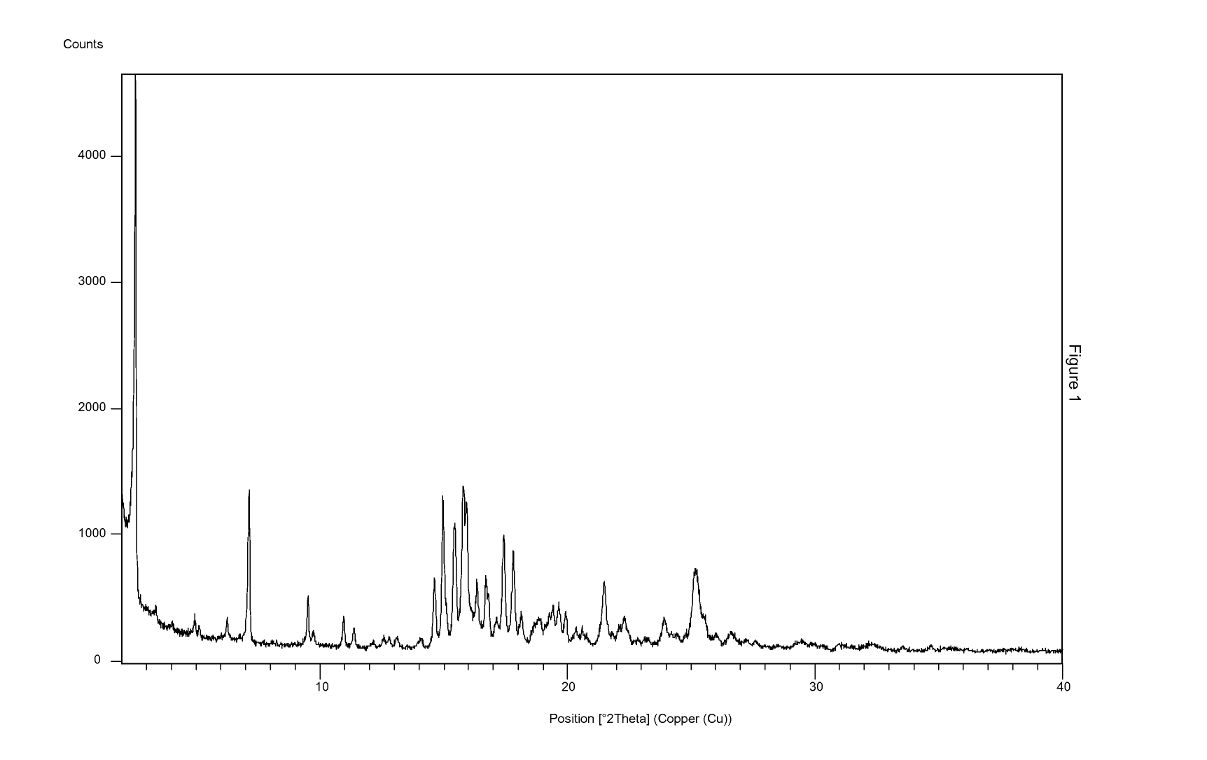

Crystalline form II-S of posaconazole can be described by an X-ray powder diffraction pattern comprising peaks at 2-theta angles of about 2.6°, 7.1°, 9.5°, 15.0°, 17.4° and 21.5°. The typical precision of the 2-theta values is in the range of ±0.2°. A characteristic X-ray powder diffraction pattern is shown in FIG. 1.

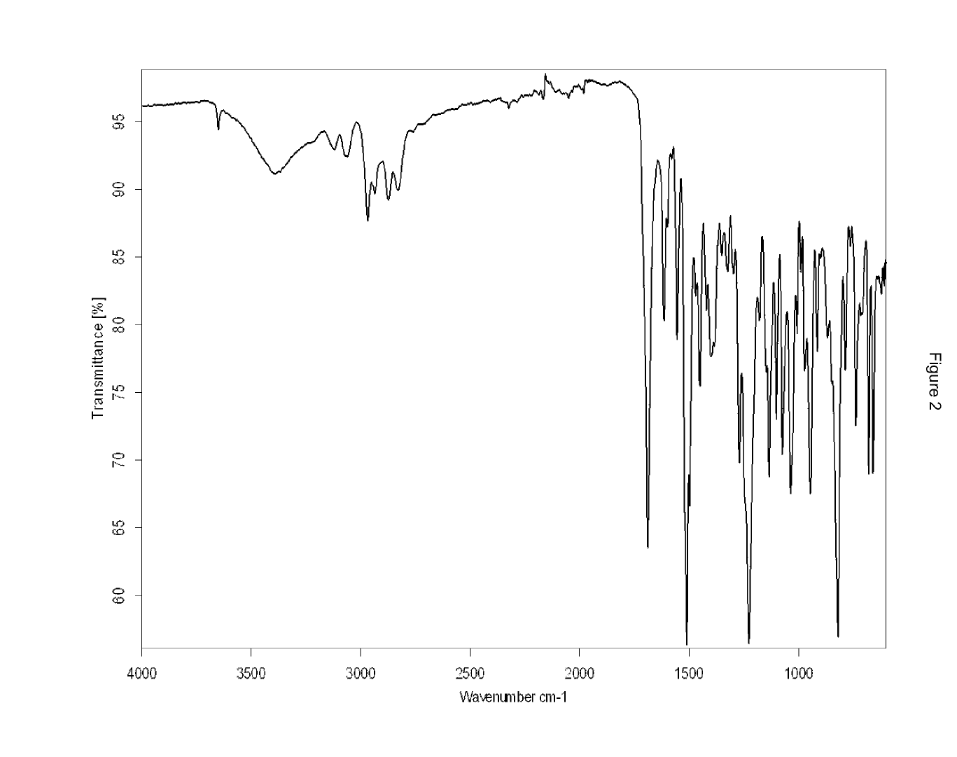

Alternatively, crystalline form II-S of posaconazole can be characterized by an attenuated total reflectance infrared spectrum comprising absorption bands at wavenumbers of about 3650 cm⁻¹, 3392 cm⁻¹, 2968 cm⁻¹, 1688 cm⁻¹, 1510 cm⁻¹, 1227 cm⁻¹, 1036 cm⁻¹, 946 cm⁻¹, 820 cm⁻¹ and 680 cm⁻¹ (±2 cm⁻¹). A typical attenuated total reflectance infrared spectrum is shown in FIG. 2.

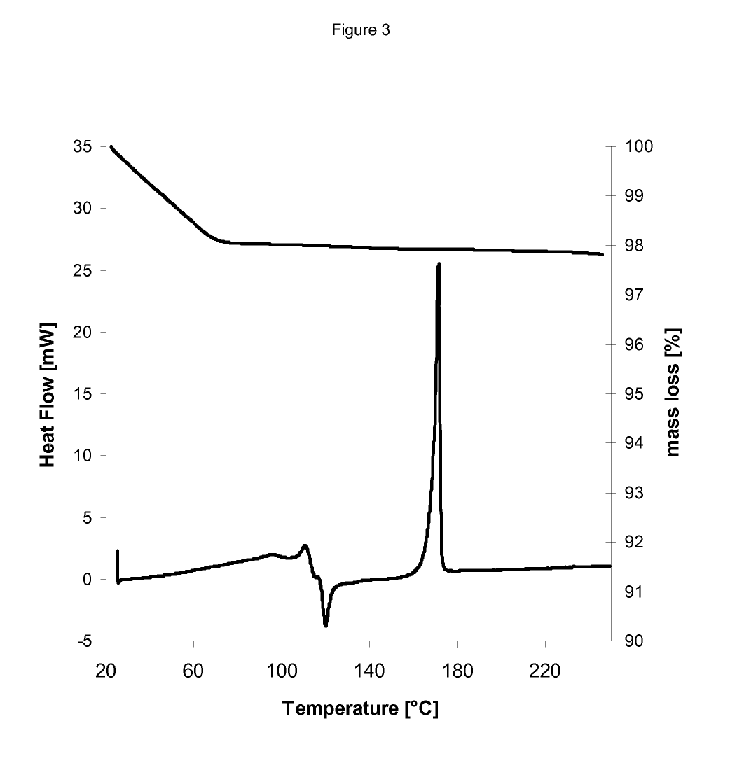

A further method for identifying crystalline form II-S is differential scanning calorimetry (DSC). A typical differential scanning calorimetry curve of crystalline form II-S can be obtained at a heating rate of 10° C./min (open pan). The DSC curve of form II-S shows a significant dehydration endotherm between 25° C. and 112° C. with peaks at 96° C. and 111° C. followed by a exothermic peak at 120.2° C.; form II-S is melting at 171.4° C. with a Tₒₙₛₑₜ of 169° C. (10° C./minute, open pan). A characteristic curve is shown in FIG. 3.

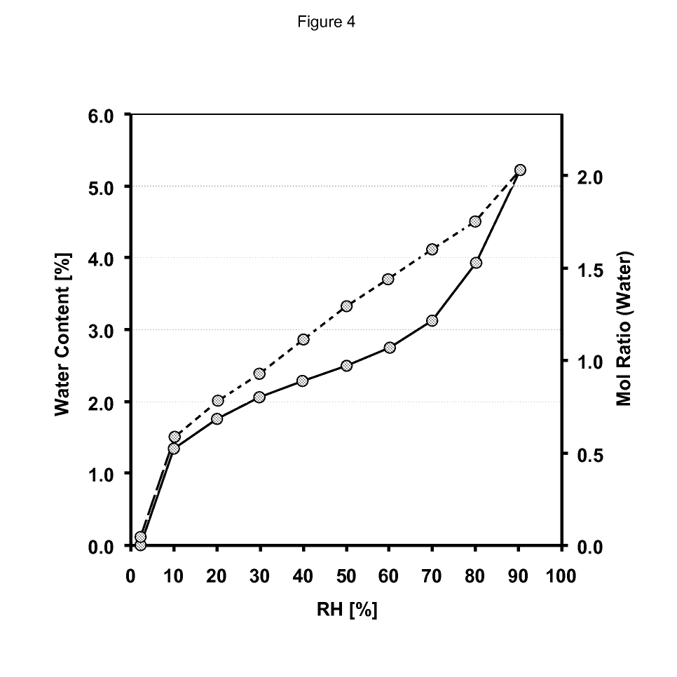

Crystalline form II-S of posaconazole contains 0 to 2.0 moles water per mole posaconazole and is thus a non-stoichiometric hydrate (see also moisture sorption isotherm of crystalline form II-S as depicted in FIG. 4). At ambient conditions, crystalline form II-S of posaconazole typically contains approximately 1 mol water per mole posaconazole which would correspond to a monohydrate.

Crystalline form II-S of posaconazole is preferably substantially pure and substantially free of other polymorphic forms or of amorphous posaconazole. Thus, crystalline form II-S of posaconazole preferably shows a polymorphic purity of at least about 90 wt.-%, preferably of at least about 95 wt.-%, e.g. of at least about 98 wt.-% as measured by XRPD analysis as herein described.

The present inventors have surprisingly found that the new crystalline form II-S of posaconazole is particularly suitable for the preparation of other polymorphic forms of posaconazole such as e.g. crystalline form II and in particular crystalline form IV of posaconazole. This finding is surprising, because these preparation processes take place within a suspension or dispersion as herein described.

Thus, crystalline form II-S of posaconazole is particularly suitable for preparing form IV of posaconazole. Form IV of posaconazole is described in co-pending European patent application no. 08159600.9.

Therefore, the present invention also relates to the use of crystalline form II-S of posaconazole for the preparation of other polymorphic crystalline forms of posaconazole, preferably of form IV of posaconazole.

Therefore, in one specific embodiment of the invention, crystalline form IV of posaconazole can be prepared by
(a′) providing a suspension or dispersion of:
   (i) posaconazole, wherein posaconazole is selected from crystalline form II-S of posaconazole; and
   (ii) a mixture of water and methanol; and
(b′) allowing the posaconazole to transform to crystalline form IV at a temperature of at most 50° C.

Crystalline form IV of posaconazole as obtained in step (b′) may be isolated and/or purified according to known methods, and/or as described in co-pending European patent application no. 08159600.9.

The posaconazole form which is used as a starting material is typically employed in the form of a powder or small crystals. The powder or small crystals may be used as such, e.g. as originating from the synthesis or may be milled or micronized before the transformation step.

The posaconazole starting material is then mixed with a mixture of water and methanol. The ratio of water to methanol (v/v) is not particularly restricted as long as the transformation results in crystalline form IV of posaconazole. Typically the ratio of water to methanol will be in the range of 20:80 to 90:10, preferably 50:50 to 85:15, more preferably 60:40 to 80:20.

The posaconazole starting material will be typically provided in a volume of a mixture of water and methanol, so that the major part is not dissolved. The mixture is typically a stirrable suspension or dispersion. The exact volume of the mixture of water and methanol will depend on the amount of methanol and the transformation conditions and can therefore vary. Typically the weight ratio of posaconazole to the mixture of water and methanol will be in the range of about 0.1 g/100 g to about 20 g/100 g, preferably from about 1 g/100 g to about 15 g/100 g, even more preferably about 2 g/100 g to about 10 g/100 g.

If desired, seed crystals of crystalline form IV of posaconazole can also be present in the mixture to aid transformation. These seed crystals may be obtained as described in co-pending European patent application no. 08159600.9.

The mixture of posaconazole, water and methanol and optionally seed crystals is then slurried, so that the posaconazole can transform to crystalline form IV.

The temperature at which the transformation can be conducted will depend on the chosen mixture of water and methanol, on the form of posaconazole which is used as a starting material, etc. Typical temperatures for conducting the transformation are about 10° C. to at most 50° C., preferably about 20° C. to about 40° C., and more preferably ambient temperature (i.e. about 20 to about 30° C.). The temperature can also vary during the transformation step. However, the suspension or dispersion is not subjected to a refluxing step during the presently claimed processes.

In a preferred embodiment a mixture of water and methanol in the ratio of 4:1 (v/v) is used in steps (a′) and (b′) of the above described processes and the preferred temperature range is about 15° C. to about 40° C.

In contrast to known processes to prepare crystalline form IV of posaconazole, the herein described processes using crystalline form II-S require slightly lower temperatures, i.e. up to at most 50° C.

The duration of the transformation step is not particularly limited. Generally, the transformation will be conducted until substantially all (e.g., preferably at least 90 wt.-%, more preferably at least 95 wt.-%) of the posaconazole starting material has been transformed into crystalline form IV. Typically the transformation step will take about 1 day or less, preferably about 7 hours to 20 hours, if seed crystals are not employed. The transformation will be quicker, e.g. from about 3 hours to about 7 hours at ambient temperature, if seed crystals are employed. If the mixture is kept at a higher temperature the speed of transformation will also be increased. A skilled person can easily determine appropriate transformation durations according to the batch size, temperature of the suspension/dispersion, presence or absence of seed crystals, etc.

The duration of the above described transformation step is shorter when compared to that of known processes to prepare crystalline form IV. In contrast to known processes, the use of crystalline form II-S of posaconazole for preparing crystalline form IV of posaconazole advantageously provides a faster process.

After the transformation step, the product, i.e. crystalline form IV of posaconazole is isolated. Transformation to crystalline form IV can be confirmed by IR or XRPD analysis as described herein.

The resultant crystalline form IV is a polymorphic form of posaconazole which is a nonstoichiometric hydrate and which is characterized by an X-ray powder diffraction pattern, an attenuated total reflectance infrared spectrum and a differential scanning calorimetry curve as described in co-pending European patent application no. 08159600.9.

Crystalline form IV of posaconazole as obtained by the process according to the invention has high purity of at least about 90 wt.-%, more preferably of at least about 95 wt.-% and most preferably of at least about 98 wt.-% as measured by XRPD analysis as herein described.

Additionally, crystalline form IV of posaconazole as obtained by the process of the invention using crystalline form II-S as starting material, has advantageous properties which make it particularly suitable for the preparation of a medicament for treating or preventing a fungal infection in a mammal, e.g. in a human patient.

Therefore, crystalline form IV of posaconazole as obtained according to the process of the present invention using crystalline form II-S as starting material, is also contemplated within the scope of the present invention. Said crystalline form IV of posaconazole may be used for treating or preventing a fungal infection.

Additionally, crystalline form II-S of posaconazole can be used to prepare crystalline form II by stirring a suspension of form II-S in a mixture of tetrahydrofurane (THF) and water, acetic acid and water or acetone and water (as is seen e.g. in Example 3). The ratio of water to tetrahydrofurane (THF) or acetic acid or acetone (v/v), as well as the weight ratio of posaconazole to the mixture of water and tetrahydrofurane (THF) or acetic acid or acetone, and the temperatures applied are similar to those described herein for the processes to prepare crystalline form IV of posaconazole.

Furthermore, crystalline form II-S of posaconazole can be used to prepare other crystalline forms of posaconazole such as crystalline forms I and III by re-crystallizing form II-S by methods known from U.S. Pat. No. 6,958,337 using a crude product.

Crystalline form II-S of posaconazole can also be used to prepare crystalline form Y which is disclosed in co-pending PCT application no. PCT/EP2009/056574 by heating crystalline form II-S at a temperature in the range of about 120° C. to about 150° C. as described in said co-pending application and as e.g. shown in Example 4.

Thus, the present invention relates to the use of crystalline form II-S of posaconazole for the preparation of other crystalline forms of posaconazole, e.g. of crystalline form I, II, III, Y or in particular of crystalline form IV of posaconazole, which can be further used as a medicament, e.g. within a pharmaceutical composition. Therefore, crystalline form II-S of posaconazole may be used to prepare other crystalline forms of posaconazole, e.g. crystalline form I, II, III, Y or in particular crystalline form IV of posaconazole, which in turn may be used for the preparation of a medicament for treating and/or preventing a fungal infection.

Alternatively, crystalline form II-S of posaconazole may also be used as a medicament as herein described.

Typical formulations and indications for posaconazole are described, for example, in WO95/17407, WO96/38443, WO02/80678, WO2005/117831, WO99/18097, U.S. Pat. No. 5,972,381, U.S. Pat. No. 5,834,472, and U.S. Pat. No. 4,957,730. It is to be noted that these patents and patent applications are given as an example only and that this list is not exhaustive. Further specific pharmaceutical compositions comprising e.g. crystalline form IV of posaconazole, are described in co-pending European patent application no. 08159600.9.

Crystalline form II-S of posaconazole is easy to prepare and offers a new additional polymorphic form for preparing antifungal medicaments. Additionally, crystalline form II-S of posaconazole is suitable for the preparation of other crystalline forms of posaconazole such as crystalline forms I, II, III, Y and IV, and thus advantageously offers a new way for preparing those forms. Moreover, crystalline form II-S is particularly useful for the preparation of crystalline form II, and is even more particularly useful to prepare crystalline form IV of posaconazole. Crystalline form II-S of posaconazole therefore provides a new way for preparing crystalline form IV of posaconazole on a commercial scale. In particular, crystalline form II-S of posaconazole provides an advantageous process for preparing crystalline form IV which is faster when compared to known processes.

The present invention is illustrated by the following examples, which should not be construed as limiting.

### EXAMPLES

The X-ray powder diffraction pattern (XRPD) was obtained with a PANalytical X'Pert PRO diffractometer equipped with a theta/theta coupled goniometer in transmission geometry, Cu-Kα_{1,2} radiation (wavelength 0.15419 nm) with a focusing mirror and a solid state PIXcel detector. The patterns were recorded at a tube voltage of 40 kV, tube current of 40 mA, applying a stepsize of 0.007° 2θ with 80 s per step (255 channels) in the angular range of 2° to 40° 2θ at ambient conditions. A typical precision of the 2-theta values is in the range of ±0.2° 2-theta. Thus a diffraction peak that appears at 5.0° 2-theta can appear between 4.8 and 5.2° 2-theta on most X-ray diffractometers under standard conditions.

Infrared spectra (IR) were collected on a MKII Golden Gate™ Single Reflection Diamond ATR (attenuated total reflection) cell with a Bruker Tensor 27 FTIR spectrometer with 4 cm⁻¹ resolution. To collect a spectrum a spatula tip of a sample was applied to the surface of the diamond in powder form. Then the sample was pressed onto the diamond with a sapphire anvil and the spectrum was recorded. A spectrum of the clean diamond was used as background spectrum. A typical precision of the wavenumber values is in the range of ±2 cm⁻¹. Thus, an infrared peak that appears at 1716 cm⁻¹ can appear between 1714 and 1718 cm⁻¹ on most infrared spectrometers under standard conditions.

Differential scanning calorimetry (DSC) was performed with a DSC 7 (Perkin-Elmer, Norwalk, Conn., USA) using the Pyris software. A sample of about 4 mg was weighed into a 25 μl Al-pan. Dry nitrogen was used as the purge gas (purge: 20 ml min⁻¹). When used herein, the term “Tₒₙₛₑₜ” determined by Differential Scanning calorimetry means the temperature corresponding to the intersection of the pretransition baseline with the extrapolated leading edge of the transition.

Thermogravimetric analysis was performed with the thermogravimetric system TGA-7 using the Pyris Software for Windows NT (Perkin-Elmer, Norwalk, Conn., USA), 50 μl platinum pans, nitrogen purge gas (sample purge: 20 ml min⁻¹, balance purge: 40 ml min⁻¹).

The moisture sorption isotherm was recorded with a SPS-11 moisture sorption analyzer (MD Mess-technik, Ulm, D). The measurement cycle was started at 0% relative humidity (RH), increased in 10% steps up to 90% RH and in a 5% step up to 95% RH. The equilibrium condition for each step was set to a constant mass±0.003% over 49 min. The temperature was 25±0.1° C.

HPLC assay was performed using the following conditions:

| | |
|---|---|
| Column | ZORBAX XDB-C18 Rapid Resolution HT, |
| | 1.8 μm, 50 × 4.6 mm (Agilent Technologies) |
| Eluent A | Dilute 2 ml of acetic acid (99-100%) with water |
| | and fill up to 1000 ml with water. Adjust pH to |
| | 6.5 with 2.5% ammonia. |
| Eluent B | water/acetonitrile = 50/50 (v/v) |
| Flow rate | 0.8 ml/min |
| Temperature | 40° C. |
| Detection | UV at 260 nm |

| | | | | |
|---|---|---|---|---|
| Gradient | t [min] | 0 | 7.5 | 20 |
| | % B | 35 | 85 | 95 |

| | |
|---|---|
| Stop time | 20 min |
| Post time | 5 min |
| Sample | about 0.5 mg/ml |
| concentration | |
| Solvent | water/acetonitrile = 50/50 (v/v) |

### Example 1

### Preparation of the Crystalline Form II-S of Posaconazole

28.8 g of Posaconazole in the form of crystalline form I prepared by the method of example 3 described in U.S. Pat. Nos. 6,958,337 and 300 ml acetone were heated under reflux in a round bottom flask provided with a reflux condenser. 90 ml water were added through the condenser and a clear solution was obtained. The hot solution was filtered and cooled down from 45° C. to 20° C. within 45 min. Crystallization started and the mixture was put in a refrigerator at 5° C. without agitation over night. The precipitated crystals were collected by filtration and then dried in a vacuum oven to yield 24.9 g of the a crystalline form of posaconazole in form of white needles

The product was analyzed by DSC, FT-IR using an attenuated total reflectance cell as herein described and XRPD and found to be a novel form of posaconazole denominated as form II-S.

Crystalline form II-S obtained according to example 1 has an X-ray powder diffraction spectrum as shown in FIG. 1. Characteristic XRPD angles, d-spacings and relative intensities are shown in Table 1.

Crystalline form II-S of posaconazole obtained above has an attenuated total reflectance IR spectrum with absorption bands at 3650, 3392, 2968, 1688, 1510, 1227, 1036, 946, 820 and 680 cm⁻¹ (±2 cm⁻¹; FIG. 2).

The obtained crystalline form II-S was subjected to differential thermal analysis. As can be seen in FIG. 3 (lower curve), crystalline form II-S shows a significant dehydration endotherm between 25° C. and 112° C. with peaks at 96° C. and 111° C. followed by a exothermic peak at 120.2° C. and melting at 171.4° C. with a Tₒₙₛₑₜ of 169° C. (10° C./minute, open pan).

At ambient conditions form II-S contains approximately 1 mol water (monohydrate). The moisture sorption isotherm of crystalline form II-S shows a distinct step over 0.5 mol water uptake/loss between 0 and 10% relative humidity. The maximum water content peaks at 5.2% at 90% relative humidity, which corresponds to a water mol ratio of 2.0 (FIG. 4).

TABLE 1
Angles 2 theta, d-values and relative intensities of form II-S

| Angle [2-Theta °] | d value [Angstrom] | Relative intensity [%] |
|---|---|---|
| 2.55 | 34.594 | 100 |
| 3.35 | 26.339 | 14 |
| 4.95 | 17.845 | 8 |
| 5.12 | 17.259 | 6 |
| 6.25 | 14.132 | 9 |
| 7.14 | 12.387 | 42 |
| 9.52 | 9.292 | 15 |
| 14.63 | 6.056 | 28 |
| 14.97 | 5.919 | 30 |
| 15.44 | 5.740 | 65 |
| 15.78 | 5.615 | 28 |
| 15.92 | 5.566 | 29 |
| 16.34 | 5.424 | 20 |
| 16.69 | 5.310 | 15 |
| 16.82 | 5.271 | 14 |
| 17.12 | 5.178 | 14 |
| 17.42 | 5.091 | 39 |
| 17.80 | 4.983 | 23 |
| 18.14 | 4.892 | 11 |
| 18.84 | 4.710 | 22 |
| 19.43 | 4.568 | 14 |
| 19.67 | 4.513 | 23 |
| 19.94 | 4.453 | 9 |
| 20.37 | 4.360 | 9 |
| 20.61 | 4.309 | 5 |
| 21.50 | 4.133 | 39 |
| 22.12 | 4.019 | 8 |
| 22.31 | 3.984 | 17 |
| 23.21 | 3.832 | 10 |
| 23.93 | 3.718 | 22 |
| 25.19 | 3.536 | 83 |
| 25.58 | 3.482 | 14 |
| 26.02 | 3.425 | 11 |
| 26.64 | 3.346 | 16 |
| 27.61 | 3.230 | 6 |
| 29.49 | 3.029 | 9 |
| 32.28 | 2.773 | 14 |

### Example 2

### Preparation of Crystalline Form IV of Posaconazole from Crystalline Form II-S of Posaconazole

8 g of posaconazole form II-S as prepared by the method of example 1 were suspended in a solution consisting of 140 ml of water and 35 ml of methanol. The mixture was stirred at 40° C. for one day. The white suspension was cooled down to room temperature and the solid was filtered and dried in vacuum overnight. The product was analyzed by XRPD and found to be 7.6 g pure crystalline form IV of posaconazole.

### Example 3

### Preparation of Crystalline Form II of Posaconazole from Crystalline Form II-S of Posaconazole

1 g of posaconazole form II-S as prepared by the method of example 1 was suspended in a solution consisting of 20 ml of water and 5 ml of acetic acid. The mixture was stirred at room temperature over night. The suspension was filtered and the solid dried in vacuum over night.

The product was analyzed by FT-IR and XRPD and found to be 0.98 g crystalline form II of posaconazole.

### Example 4

### Preparation of Crystalline Form Y of Posaconazole from Crystalline Form II-S of Posaconazole

10.94 g of crystalline form II-S of posaconazole as prepared by the method of example 1 was placed in a 140° C. preheated Büchi Kugelrohr apparatus and agitated for 15 minutes. The anhydrous product obtained was cooled and analyzed by XRPD and IR. The product obtained was the anhydrous polymorphic form Y of posaconazole.

### Example 5

### Preparation of Crystalline Form III of Posaconazole from Crystalline Form II-S of Posaconazole

4.89 g of crystalline form II-S of posaconazole as prepared by the method of example 1 in 98 ml of methanol was heated up until a clear solution was obtained. The solution was cooled down to 25° C. Afterwards the mixture was cooled down from 25° C. to 15° C. within 20-40 min. The product began to crystallize out of solution at this temperature. The reaction mixture was held at 15° C. for 30 min. The slurry was cooled to 0° C. over 30 min and hold for an additional hour. The precipitated crystals were collected and dried to provide 3.97 g of polymorphic form III of posaconazole.

1. Crystalline form II-S of posaconazole having an X-ray powder diffraction pattern comprising peaks at 2-theta angles of about 2.6°±0.2°, 7.1°±0.2°, 9.5°±0.2°, 15.0°±0.2°, 17.4°±0.2° and 21.5°±0.2°.

2. The crystalline form II-S of posaconazole according to claim 1, wherein the crystalline form II-S has an X-ray powder diffraction pattern substantially in accordance with FIG. 1.

3. Crystalline form II-S of posaconazole having an attenuated total reflectance infrared spectrum comprising absorption bands at wavenumbers of about 3650 cm⁻¹±2 cm⁻¹, 3392 cm⁻¹±2 cm⁻¹, 2968 cm⁻¹±2 cm⁻¹, 1688 cm⁻¹±2 cm⁻¹, 1510 cm⁻¹±2 cm⁻¹, 1227 cm⁻¹±2 cm⁻¹, 1036 cm⁻¹±2 cm⁻¹, 946 cm⁻¹±2 cm⁻¹, 820 cm⁻¹±2 cm⁻¹ and 680 cm⁻¹±2 cm⁻¹.

4. The crystalline form II-S of posaconazole according to claim 3, wherein the crystalline form II-S has an attenuated total reflectance infrared spectrum substantially in accordance with FIG. 2.

5. Crystalline form II-S having a differential scanning calorimetry curve substantially in accordance with that shown in FIG. 3.

6. Crystalline form II-S of posaconazole containing 0 to 2.0 moles water per mole posaconazole.

7. A process for the preparation of crystalline form II-S of posaconazole comprising the following steps:
(a) admixing posaconazole and acetone in a concentration of posaconazole of about 1 g per 10 ml of acetone and heating the obtained mixture under reflux;
(b) adding water to the mixture obtained in step (a) while maintaining at reflux temperature to obtain a solution which contains acetone and water in a volume/volume (v/v) ratio of 10:3;
(c) cooling the solution obtained in step (b) to 0° C.-5° C. within a time period of about 1 to 2 hours to obtain a suspension; and
(d) isolating the solid product formed in the suspension obtained in step (c) by filtration.

8. A process for the preparation of crystalline form IV of posaconazole comprising the steps of:
(a′) providing a suspension or dispersion of:
(i) crystalline form II-S of posaconazole; and
(ii) a mixture of water and methanol; and
(b′) transforming the crystalline form II-S of posaconazole into crystalline form IV at a temperature of at most 50° C.

9. A method of using crystalline form II-S of posaconazole having an X-ray powder diffraction pattern comprising peaks at 2-theta angles of about 2.6°±0.2°, 7.1°±0.2°, 9.5°±0.2°, 15.0°±0.2°, 17.4°±0.2° and 21.5°±0.2°, the method comprising the step of converting the crystalline form II-S of posaconazole into another crystalline form of posaconazole.

10. A method of using crystalline form II-S having an X-ray powder diffraction pattern comprising peaks at 2-theta angles of about 2.6°±0.2°, 7.1°±0.2°, 9.5°±0.2°, 15.0°±0.2°, 17.4°±0.2° and 21.5°±0.2°, the method comprising the step of using crystalline form II-S for the preparation of a crystalline form of posaconazole which is used as a medicament.

11. A method of using crystalline form II-S having an X-ray powder diffraction pattern comprising peaks at 2-theta angles of about 2.6°±0.2°, 7.1°±0.2°, 9.5°±0.2°, 15.0°±0.2°, 17.4°±0.2° and 21.5°±0.2°, the method comprising the step of using crystalline form II-S to prepare a crystalline form of posaconazole which is used for the preparation of a medicament for treating or preventing a fungal infection.

12. The method of using crystalline form II-S of posaconazole use according to claim 9, wherein the another crystalline form of posaconazole is crystalline form IV of posaconazole.

13. Crystalline form IV obtained according to a process for the preparation of crystalline form IV of posaconazole comprising the steps of:
(a′) providing a suspension or dispersion of:
(i) crystalline form II-S of posaconazole; and
(ii) a mixture of water and methanol; and
(b′) transforming the crystalline form II-S of posaconazole into to transform to crystalline form IV at a temperature of at most 50° C.

14. A method of using crystalline form II-S of posaconazole having an attenuated total reflectance infrared spectrum comprising absorption bands at wavenumbers of about 3650 cm⁻¹±2 cm⁻¹, 3392 cm⁻¹±2 cm⁻¹, 2968 cm⁻¹±2 cm⁻¹, 1688 cm⁻¹±2 cm⁻¹, 1510 cm⁻¹±2 cm⁻¹, 1227 cm⁻¹±2 cm⁻¹, 1036 cm⁻¹±2 cm⁻¹, 946 cm⁻¹±2 cm⁻¹, 820 cm⁻¹±2 cm⁻¹ and 680 cm⁻¹±2 cm⁻¹, the method comprising the step of converting the crystalline form II-S of posaconazole into another crystalline form of posaconazole.

15. The method of using crystalline form II-S of posaconazole according to claim 14, wherein the another crystalline form of posaconazole is crystalline form IV of posaconazole.

16. A method of using crystalline form II-S having an attenuated total reflectance infrared spectrum comprising absorption bands at wavenumbers of about 3650 cm⁻¹±2 cm⁻¹, 3392 cm⁻¹±2 cm⁻¹, 2968 cm⁻¹±2 cm⁻¹, 1688 cm⁻¹±2 cm⁻¹, 1510 cm⁻¹±2 cm⁻¹, 1227 cm⁻¹±2 cm⁻¹, 1036 cm⁻¹±2 cm⁻¹, 946 cm⁻¹±2 cm⁻¹, 820 cm⁻¹±2 cm⁻¹ and 680 cm⁻¹±2 cm⁻¹, the method comprising the step of using crystalline form II-S for the preparation of a crystalline form of posaconazole which is used as a medicament.

17. A method of using crystalline form II-S having an attenuated total reflectance infrared spectrum comprising absorption bands at wavenumbers of about 3650 cm⁻¹±2 cm⁻¹, 3392 cm⁻¹±2 cm⁻¹, 2968 cm⁻¹±2 cm⁻¹, 1688 cm⁻¹±2 cm⁻¹, 1510 cm⁻¹±2 cm⁻¹, 1227 cm⁻¹±2 cm⁻¹, 1036 cm⁻¹±2 cm⁻¹, 946 cm⁻¹±2 cm⁻¹, 820 cm⁻¹±2 cm⁻¹ and 680 cm⁻¹±2 cm⁻¹, the method comprising the step of using crystalline form II-S to prepare a crystalline form of posaconazole which is used for the preparation of a medicament for treating or preventing a fungal infection.

18. The method of using crystalline form II-S of posaconazole according to claim 10, wherein the crystalline form of posaconazole is crystalline form IV of posaconazole.

19. The method of using crystalline form II-S of posaconazole according to claim 11, wherein the crystalline form of posaconazole is crystalline form IV of posaconazole.

20. The method of using crystalline form II-S of posaconazole according to claim 14, wherein the another crystalline form of posaconazole is crystalline form IV of posaconazole.

21. The method of using crystalline form II-S of posaconazole according to claim 16, wherein the crystalline form of posaconazole is crystalline form IV of posaconazole.

22. The method of using crystalline form II-S of posaconazole according to claim 17, wherein the crystalline form of posaconazole is crystalline form IV of posaconazole.
